(19) United States
(12) Patent Application Publication
(10) Pub. No.: US 2005/0147581 A1
(43) Pub. Date: Jul. 7, 2005

(54) Macromolecular drug complexes having improved stability and therapeutic use of the same

(75) Inventors: Camellia Zamiri, Fremont, CA (US); Michael J. Groves, Deerfield, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE BOARD OF TRUSTEES OF THE UNIVERSITY OF ILLINOIS, Urbana, IL (US)

(21) Appl. No.: 10/987,503

(22) Filed: Nov. 12, 2004

Related U.S. Application Data
(60) Provisional application No. 60/523,211, filed on Nov. 19, 2003.

(51) Int. Cl. A61K 38/28; A61K 38/20; A61K 38/21; A61K 38/26; A61K 31/785

(52) U.S. Cl. 424/78.27; 424/130.1; 530/391.1; 514/12; 514/3; 424/85.4; 514/28; 514/62; 525/54.1; 525/54.2

(57) ABSTRACT

Macromolecular drug complexes containing a protein therapeutic, like human growth hormone, and an excess stoichiometric molar amount of a polymer, like heparin, and compositions containing the same, are disclosed. Compositions containing the macromolecular drug complexes are administered, including via pulmonary delivery, to individuals suffering from a disease or condition, and the complexes release the protein therapeutic, in vivo, to treat the disease or condition.

### CROSS REFERENCE TO RELATED APPLICATIONS

This application claims the benefit of provisional U.S. patent application Ser. No. 60/523,211, filed Nov. 19, 2003.

### FIELD OF THE INVENTION

The present invention relates to macromolecular drug complexes and to the administration of compositions containing a present macromolecular drug complex to an individual in need thereof. More particularly, the present invention relates to a macromolecular drug complex containing a protein therapeutic, like human growth hormone (hGH), that is noncovalently bound, i.e., is complexed, to a polymer having a plurality of acid moieties, like heparin. The stability of the macromolecular drug complex is enhanced by utilizing a molar amount of the polymer in excess of the stoichiometric molar amount required to complex with the protein therapeutic. The macromolecular drug complex is incorporated into a pharmaceutical formulation for administration of the protein therapeutic, including the pulmonary administration of the complex in a formulation further comprising chitosan.

### BACKGROUND OF THE INVENTION

It is well known that modern day drugs are very efficacious with respect to treating acute and chronic diseases. For example, the standard treatment for diabetes is administration of insulin. An individual suffering from diabetes does not produce sufficient insulin, thus the individual cannot burn and store glucose. Diabetes cannot be cured, but diabetes can be treated by periodic injections of insulin. In mild diabetics, the rise in serum insulin is lower compared to normal individuals. In severe diabetics, no insulin is produced, and the rise in serum insulin levels is negligible. As a result, excess glucose accumulates in the blood of a diabetic, which can result, for example, in a loss of weight and loss of strength.

A serious disadvantage with respect to present-day therapeutic compositions used to treat diabetes is that insulin must be injected. Insulin cannot be administered orally because insulin is destroyed by the strong acid conditions of the stomach. Similarly, other protein therapeutics, like hGH, must be injected because they also are destroyed by the strong acid conditions in the stomach, and cannot be administered orally.

Somatropin, also termed human growth hormone (hGH), is a protein drug (22 kDa) successfully administered to children with growth failure attributed to an inadequate secretion of endogenous growth hormone, and to adults as a replacement therapy. Somatropin is administered by subcutaneous injections, six or seven times per week, for years. Somatropin possesses many of the disadvantages of other proteinaceous drugs, including short in vivo half-life (20 minutes), because of physical and chemical instabilities and enzymic degradation, which also makes somatropin unstable in vitro.

In order to overcome the pain of injection, increase compliance, and to improve the quality of life, investigators have strived to devise noninvasive somatropin delivery systems. The lung is one relatively unexploited route of delivery for large therapeutic molecules that would otherwise must be administered by injection. Previous studies have shown that the lung provides substantially more absorption sites for macromolecules than any other port of entry to the body, probably due to a high internal surface area.

Therefore, it would be advantageous to provide compositions based on a protein therapeutic to treat a disease or condition, and it also would be advantageous to develop easier methods of administering a protein therapeutic to an individual. It particularly would be advantageous to stabilize somatropin and provide compositions that facilitate the absorption of somatropin via the pulmonary route. As set forth in detail hereafter, the present invention is directed to macromolecular drug complexes containing a protein therapeutic and having improved stability, to pharmaceutical formulations containing the complexes, and to use of the stabilized complexes to treat a disease or condition. The present invention is further directed to improved drug delivery to facilitate administration of difficult-to-administer drugs, like insulin and hGH, including pulmonary administration.

With respect to diabetes, glycosaminoglycans (GAGs) are a class of negatively charged, endogenous polysaccharides composed of repeating sugar residues (i.e., uranic acids and hexosamines). GAGs are known to bind a variety of biological macromolecules, including connective tissue macromolecules, plasma proteins, lysosomal enzymes, and lipoproteins. In addition, exogenous GAGs have been shown to bind to the cell surfaces of a variety of different cell types, including liver cells (i.e., hepatocytes), fibroblasts, and importantly, endothelial cells. Exogenous GAGs, therefore, can be internalized. Furthermore, GAGs have been (a) implicated in the regulation of cell proliferation and in cell-cell communication, (b) shown to interact with cell-surface receptors (cell adhesion molecules), and (c) shown to modify the behavior of cells in culture. In addition, GAGs were shown to be highly potent and selective inhibitors of HIV replication and giant cell formation.

GAG-receptor interactions are characterized by the formation of noncovalent, self-assembling macromolecular complexes. These transient, interpolyelectrolyte complexes mediate many biological functions including enzyme-substrate binding, antigen-antibody interactions, leukocyte-endothelial cell adhesion events, drug-receptor binding, and protein-protein interactions. Furthermore, secondary binding forces, such as hydrogen bonds, van der Waals forces, and hydrophobic interactions, govern interpolyelectrolyte formation, and, ultimately, influence the resulting pharmacologic response to the complex.

G. Gambaro et al., Kidney Int., 46, pages 797-806 (1994) discloses that exogenously administered GAGs have a favorable effect on morphological and functional renal abnormalities in diabetic rats, and appear to revert established diabetic renal lesions. D. M. Templeton, Lab. Invest., 61(2), pages 202-211 (1989) and C. W. Marano et al., Invest. Ophthalmology Vis. Sci., 33(9), pages 2619-2625 (1992) disclose that diabetic patients have a decreased glycosaminoglycan content in glomerular basement membranes. Additionally, an increase in total GAG serum levels in diabetic patients was disclosed in K. Olczyk et al., Acta Biochimica Polonica, 39, pages 101-105 (1992). The authors observed an increase in protein-bound GAGs, such as keratan sulfate, hyaluronic acid, heparin sulfate, and heparin, in diabetic patients. The Gambaro et al. publication also discloses an increase in the urinary excretion rate of GAGs from insulin-dependent diabetic patients.

Therefore, research has shown that glycosaminoglycans play an important, yet unexplained, role in the vascular changes associated with lifelong insulin therapy. In particular, administration of GAGs to diabetic animals has inhibited or reversed some vascular abnormalities. The publications also strongly suggest that exogenous insulin plays a role in elevating the level of GAGs in the urine and serum of diabetic patients. Furthermore, the literature clearly shows that glycosaminoglycans bind to a multitude of biological macromolecules, including proteins.

These observations appear to suggest utilizing glycosaminoglycans as an adjuvant to insulin therapy. However, GAGs are anticoagulants and long term use of GAGs with insulin may thin the blood of an individual. The risks associated with a long-term use of GAGs also are unknown. Although GAGs have been used as therapeutic agents, e.g., heparin, GAGs typically have not been used for extended periods of time, or in the treatment of a chronic disease or condition, like diabetes or dwarfism. The present invention is directed to stabilized drug complexes, and compositions containing the stabilized drug complexes, that provide the benefits of a drug GAGs complex, but that avoid the disadvantages associated with long term administration of a GAG compound.

U.S. Pat. No. 6,417,237 discloses a macromolecular drug complex comprising a drug having at least one quaternary ammonium ion, such as insulin or hGH, and a polymer having a plurality of acid moieties, including synthetic and naturally occurring polymers, like heparin. However, hGH, insulin, and other protein therapeutics are susceptible to a variety of degradation processes. Investigators have sought methods and compositions to improve the stability of such protein therapeutics. Investigators also have sought routes of administration different from injection in order to improve patient compliance during a chronic treatment regimen. The present invention is directed, in part, to improving the stability of protein therapeutics, and to facilitating administration of protein therapeutics.

### SUMMARY OF THE INVENTION

The present invention is directed to macromolecular drug complexes containing a protein therapeutic and having improved stability. The present invention also is directed to pharmaceutical formulations containing a stabilized macromolecular drug complex and to methods of administering a stabilized macromolecular drug complex, including pulmonary administration.

The stabilized macromolecular drug complexes treat (a) the underlying disease or condition, e.g., insulin to treat diabetes or human growth hormone to treat dwarfism, hypopituitarism, hypercholesterolemia, hypertension, depression, muscle wasting, osteoporosis, insomnia, menopause, impotence, as well as other conditions commonly associated with aging, and (b) complications associated with the disease or condition, e.g., prevent or reverse the vascular problems associated with diabetes.

More particularly, the present invention is directed to a macromolecular drug comprising a protein therapeutic and a polymer having a plurality of acid moieties, such as heparin (UH, unfractionated heparin), having a weight average molecular weight (M_{w}) of about 1,000 to about 50,000. In accordance with an important aspect of the present invention, the protein therapeutic is a polypeptide, protein, or mixture thereof.

Another aspect of the present invention is to provide a macromolecular drug complex wherein the polymer is a naturally occurring polymer that is present in a molar amount in excess of the stoichiometric amount required to complex the protein therapeutic. As illustrated hereafter, the molar stoichiometric amount of a polymer required to complex with a protein therapeutic typically is different from, but can be, a 1:1 molar ratio. The molar stoichiometric amount of a polymer required for complexing with a protein therapeutic is readily determined by persons skilled in the art using standard techniques.

Another aspect of the present invention is to provide a pharmaceutical formulation comprising a stabilized macromolecular drug complex of the present invention and chitosan, particularly chitosan microparticles. The pharmaceutical formulation can be administered to an individual to treat an acute or chronic disease or condition, and to alleviate, eliminate, or reverse complications associated with the disease. The pharmaceutical formulation can be administered by a variety of routes, including pulmonary administration.

Another aspect of the present invention is to provide a macromolecule drug complex that remains intact and does not dissociate immediately after administration, and that is capable of releasing a protein therapeutic in vivo to treat a disease or condition.

Still another aspect of the present invention is to provide a stabilized macromolecular drug complex wherein the drug is human growth hormone, insulin, a polypeptide therapeutic, a protein therapeutic, or a mixture thereof.

Another aspect of the present invention is to provide a stabilized macromolecular drug complex comprising human growth hormone and a naturally occurring polymer containing a plurality of acid moieties, like heparin wherein the complex contains an excess of a molar amount of the polymer required to complex with the hGH.

Yet another aspect of the present invention is to provide a stabilized macromolecular human growth hormone complex that treats dwarfism, hypopituitarism, hypercholesterolemia, hypertension, depression, muscle wasting, osteoporosis, insomnia, menopause, impotence, as well as other conditions commonly associated with aging.

One other aspect of the present invention is to provide alternate routes of administration for the safe, easy, and effective delivery of a protein therapeutic agent, especially to provide a pulmonary route of administration for insulin, human growth hormone, and other protein therapeutics.

These and other novel features and aspects of the present invention will become apparent from the following detailed description of the preferred embodiments.

### BRIEF DESCRIPTION OF THE DRAWINGS

FIG. 1 contains plots of optical density (OD) vs. agitation time (sec) for the precipitation of hGH and hGH/UH complexes at different pH values;

FIGS. 2 and 3 contain plots of hGH vs. time (days) for the amount of hGH remaining in solution after 93 days storage at 4° C. and 37° C., respectively;

FIGS. 4 and 5 contain plots of cumulative body weight gain (in grams) vs. time (days) after daily subcutaneous or alternate daily intratracheal administration of hGH and hGH/UH complexes to hypophysectomized rats, respectively;

FIG. 6 contains plots of cumulative body weight gain (grams) vs. time (days) for alternate daily intratracheal administration of hGH and hGH/UH complexes with chitosan;

FIG. 7 contains bar graphs of normalized cumulative weight gain over 10 days (grams) vs. chitosan amount (mg/kg) for alternate daily intratracheal administration of hGH/UH complexes and chitosan to hypophysectomized rats; and

FIG. 8 contains a plot of normalized growth rate (g/day) vs. chitosan amount (mg/kg) for administration of hGH/UH complexes and chitosan particles to hypophysectomized rats.

### DETAILED DESCRIPTION OF THE PREFERRED EMBODIMENTS

Administration of hGH is a known and successful treatment for dwarfism in children, who would otherwise would be growth retarded. hGH presently is administered by subcutaneous injections, mainly to growth hormone deficient children, at 0.025 to 0.05 mg/kg body weight, daily or six times per week. hGH has the disadvantages of other protein therapeutics, such as a short in vivo half-life (i.e., 20 minutes) attributed to physical and chemical instability and enzymic degradation. The pain and inconvenience of injections, especially in children, has resulted in an extensive search for noninvasive routes for hGH delivery.

hGH is susceptible to a variety of degradation process including deamidation, oxidation, reduction, aggregation, and hydrolysis. Commercial hGH freeze-dried formulations (i.e., formulations containing glycine and mannitol as bulking agents to maintain good cake structure and decrease the duration of the lyophilization cycle) have a shelf life of two years at 2° C. to 8° C. Once reconstituted, the resulting solution is stable for about two weeks at 2° C. to 8° C., and must contain a preservative if multiple injections are contemplated (R. Pearlman et al., 1993).

Substantial research has been directed to improving the stability of hGH. Katakam et al. (1997) used poloxamer polymers to stabilize hGH from various processing stresses, such as air/water inerfaces, adsorption to hydrophobic surfaces, and temperature. Poloxamer 407 was found to be an effective hGH stabilizer for protection against interfacial and thermal stress. However, the biological activity of the stabilized formulation was not tested, and no long-term stability improvements attributed to poloxamers have been reported.

Bam et al. (1998) reported the effects of TWEEN® surfactants on inhibition of hGH aggregation against agitation-induced damage through hydrophobic interactions. The stabilizing effect does not correlate with the critical micelle concentration (cmc) of the surfactant, but rather the amount of surfactant required to saturate the hydrophobic sites. The amount of surfactant required to prevent hGH aggregation also is high (i.e., 225-1620 μM), which makes the therapeutic delivery of such formulations quite problematic for these surfactant-containing formulations. Moreover, no in vivo data was reported.

Human growth hormone pretreated with zinc salt, and optionally lysine or calcium ions, was dislosed as providing a high stability against deamidation, oxidation, and cleavage of peptide bonds (Sorensen et al., 2000). Although zinc poisoning in man has not been identified with certainty, prolonged zinc use may lead to copper deficiency and anemia. Zinc sulfate also can be converted to corrosive zinc chloride, and it is this corrosive action that accounts for the acute toxicity of the soluble zinc salts.

The use of heparin as a stabilizing agent for growth factors, such as acidic fibroblast growth factor (a-FGF), keratinocyte growth factor (KGF), and transforming growth factor-beta 2 (TFG-β2), has been reported. Transforming growth factor-beta 2 (TGF-β2) is a protein for the treatment of chronic skin ulcers and multiple sclerosis. Heparin (Hep) has been reported as a stabilizing agent for TGF-β2 by Schroeder-Tefft et al. (1997). TGF-β2 loses biological activity under physiological conditions as measured by loss of activity in PBS at pH 7.4 and 37° C. In vitro studies showed that Hep/TGF-β2 remained active, whereas TGF-β2 alone lost activity, when stored for two months in PBS at pH 7.4 and 37° C.

The present invention demonstrates that the stability of hGH is enhanced in the presence of an excess stoichiometric complexing amount of heparin, without changes in biological activity, and without restrictions in modes of delivery of the protein therapeutic. This improved stability provides easier handling of hGH during the preparation, sterilization, shipping, and storage processes. The present invention represents an important advance in the art of production and delivery of protein therapeutics because a stabilized form of the protein therapeutic is provided, and administration to a patient with fewer handling restrictions and precautions is achieved, which increases patient compliance.

In a particularly preferred embodiment, hGH is complexed with an excess stoichiometric molar complexing amount of heparin. Heparin is the preferred GAGs for complexing to hGH because:
a) heparin is the most highly charged polyanion in nature, and has the highest binding strength among GAGs for interacting with proteins;
b) heparin is biocompatible, biodegradable, and nonimmunogenic, and degrades in the body to toxicologically acceptable products;
c) heparin is very stable, losing only about one-half of its original anticoagulant activity after 12 years storage at 37° C.;
d) heparin is readily available and inexpensive;
e) the effectiveness of heparin as a stabilizing agent for growth factors has been confirmed; and
f) hGH is a candidate for patients with AIDS-related wasting syndrome because of its anabolic effects that increases protein synthesis and has anticatabolic effects.

The use of polymers in drug delivery systems is well established. For example, polymers, such as polylactic glycolic acid (PLGA), have been reported as vehicles for sustained release of hGH. However, several formulations have the disadvantages of requiring organic solvents, or shear stress or high temperature during preparation, which adversely affects hGH structure and bioactivity. In addition, the majority of previously used polymers did not offer alternative routes of administration for hGH, besides parenteral, where pain and inconvenience remains a problem. In addition, in children, the longitudinal growth response to hGH replacement therapy was greater when the identical dose was administered in divided doses three to four times weekly, rather than once per week due to simulating pulsatile secretion of endogenous hGH (Frasier, 1983). Therefore, controlled release formulations of hGH, such as PLGA microspheres (NUTROPIN DEPOT® from Genentech, which is administered parenterally once or twice per month), may not be an ideal formulation for producing the maximum clinical response to hGH in humans. Attention also should be drawn to the need for delivering hGH over shorter periods.

The lungs represent a relatively unexploited route of delivery for large therapeutic molecules, like protein therapeutics, that otherwise are delivered by injection. Studies have shown that in the absence of surfactant enhancers, the lungs provide substantially greater bioavailability for macromolecules than any other port of entry to the body (Patton et al., 1992). Relative to subcutaneous injection, bioavailabilities of small peptides and insulin (i.e., <6 kDa) placed into the lungs can approach or attain 100%. Furthermore, larger proteins (i.e., 18-22 kDa), such as granulocyte colony stimulating factor (GCSF), interferon α, and hGH, have exhibited pulmonary bioavailability approaching or exceeding 50% relative to subcutaneous injection (Patton et al. 1989-1990). High lung bioavailability is theorized to stem from immediate access to a large surface area (e.g., about 100 m²) provided by pulmonary delivery and/or slow clearance from the deep lung. The lungs also exhibit significant extracellular protease inhibitory activity (Patton, 1996). Thin alveolar epithelium, extensive vascularization, and lack of hepatic first pass metabolism are additional advantages of pulmonary delivery.

Diketopiperazine polymers have been reported as pulmonary delivery systems for proteins (i.e., insulin and calcitonin) (Steiner et al., 2002). The main disadvantage of these delivery systems is the use of organic solvents. Also, no data is available comparing pulmonary deliveries of protein alone and with diketopiperazine microparticles, either in U.S. Pat. No. 6,428,771 or in the literature.

Chitosan is a linear polysaccharide comprised of two monosaccharides, i.e., N-acetyl-D-glycosamine and D-glucosamine, linked together by β (1-4) glucosidic bonds (Singla et al., 2001). Possible biological applications of chitosan include cholesterol lowering, wound healing, and haemostatic and antimicrobial activity.

Chitosan has been studied extensively as a drug-delivery system in controlled release formulations and colon targeting. Chitosan has the advantages of being biocompatible, and being a biodegradable absorption enhancer and mucoadhesive. Chitosan is nontoxic, having an oral LD₅₀ in mice in excess of 16 g/kg (Arai et al., 1968). The mucoadhesive properties of chitosan primarily are attributed to the cationic nature of chitosan, which can provide a strong electrostatic interaction with the negatively charged mucus glycoprotein (He et al., 1998). In addition to mucoadhesion, chitosan has been shown to enhance drug absorption via the paracellular route (Artursson et al., 1994). It is theorized, but not relied upon herein, that because the lungs also covered by a mucus layer, chitosan has a potential as a pulmonary delivery system, facilitating the passage of large molecules, such as hGH-heparin complexes across the pulmonary mucosa.

Successful use of chitosan in the nasal delivery of peptides and proteins, such as albumin, interleukins, insulin, and human growth hormone, has been reported (Illum et al., 1994; Witschi et al., 1999). U.S. Pat. Nos. 5,690,954 and 5,863,554 disclose a chitosan microsphere preparation for the nasal delivery of peptides and proteins including hGH. However, chitosan microspheres were not effective alone and the presence of an absorption enhancing material, such as a phospholipid, was required to provide improved effects. In addition, the size of the chitosan microspheres was about 10 to about 90 μm, which is outside the range (i.e., about 1-5 μm) for pulmonary delivery. Moreover, bioadhesive microspheres, such as chitosan, are used to administer proteins only to the nose, eye, and vagina.

The present invention utilizes chitosan to improve the pulmonary delivery of a present hGH-polymer complex, in particular because the lungs not only have a mucus layer, but also provide a higher surface area and vasculature than the nose for the absorption of protein therapeutics.

Yamamoto et al. (2000) reported the pulmonary delivery of the peptide elcatonin via surface modification of lactide/glycolide copolymer nanospheres with chitosan. The results indicated improved pulmonary delivery of elcatonin with chitosan-nanospheres compared to drug alone. However, the results were incomplete, and, therefore, conclusions based on their current results are not reliable.

hGH is a complex protein hormone that is readily denatured by the shear forces experienced during administration to the lung. A stabilized hGH/heparin macromolecular complex of the present invention overcomes this problem, but still requires a delivery vehicle. The present vehicle, chitosan, is theorized to be a mucoadhesive and absorption enhancer, and can be credited with enhancement of biological activity (i.e., weight gain of hypophysectomized rats) at a critical concentration. The present invention, therefore, is an unexpected advance in the art for the pulmonary delivery of hGH.

In particular, the present invention is directed to a macromolecular drug complex containing a protein therapeutic and a naturally occurring polymer having a plurality of acid moieties, wherein the drug complex contains a molar excess of the polymer over the stoichiometric molar amount needed to complex with the protein therapeutic. The excess polymer provides a stabilized macromolecular drug complex. The stabilized macromolecular drug complex is useful for the oral, parenteral, buccal, sublingual, transdermal, conjunctival, intraocular, intranasal, aural, intrarespiratory, rectal, vaginal, or urethral delivery of protein therapeutic. When admixed with chitosan in a pharmaceutical formulation, a stabilized macromolecular drug complex can be administered by pulmonary delivery. The protein therapeutic is a polypeptide or a protein. In especially preferred embodiments, the protein therapeutic is human growth hormone or insulin.

The following discussion is particularly directed to the preparation, characterization, and evaluation of stabilized macromolecular drug complexes including human growth hormone (as the protein therapeutic) and heparin (as the polymer). A present macromolecular drug complex is prepared from a mixture of a protein therapeutic agent and an excess stoichiometric molar amount of a polymer containing a plurality of acid moieties. Once formed, a stabilized macromolecular drug complex can be incorporated, for example, into the dispersed phase or continuous phase of an oil-in-water (O/W) or water-in-oil (W/O) microemulsion, respectively. The microemulsions containing the macromolecular drug complex then can be administered by a variety of routes, including oral and parenteral, as set forth in U.S. Pat. No. 6,417,237, incorporated herein by reference. When admixed with chitosan in a pharmaceutical formulation, the stabilized macromolecular drug complex is suitable for pulmonary delivery.

Complexation of human growth hormone (hGH), a 22 kD (kilodalton) protein, with heparin, which is an endogenous anionic polysaccharide, was confirmed visually and by turbidimetry. In particular, visually clear, aqueous solutions of an acidic solution growth hormone and heparinic acid, made by passage of sodium heparin through an acidic ion exchange resin, were admixed. The immediate formation of an opalescent colloidal solution indicated the formation of the growth hormone-heparin complex. Turbidimetric analysis of the resulting colloidal solution indicated that the pH of the complexing medium influences the particle size and composition of the complex. Formulation of the hGH-heparin complex with chitosan, and subsequent pulmonary administration, enhanced hGH absorption.

Persons skilled in the art are aware that other protein therapeutics similarly can be complexed with an excess stoichiometric molar amount of a polymer having a plurality of acid moieties to provide a stabilized macromolecular drug complex.

An advantage of the present invention is to provide human growth hormone in a form capable of treating diseases and conditions such as dwarfism, hypopituitarism, hypercholesterolemia, depression, muscle wasting, osteoporosis, insomnia, menopause, impotence, as well as other conditions commonly associated with aging. The effect of human growth hormone in treating these diseases and conditions is set forth in the following table:

| Condition | Action of Growth Hormone |
|---|---|
| Dwarfism | stimulates osteoblast production |
| Muscle wasting | enhances lean muscle mass and reduces body fat |
| (AIDS) | through improved protein synthesis |
| Hypercholesterolemia | reduces cholesterol (lowers LDL) |
| Osteoporosis | enhances bone density through the stimulation of |
| | osteoblast growth |
| Autoimmune disorders | enhances immune system efficiency |
| Depression | alleviates the symptoms and syndromes associated |
| | with depression through its mood-elevating |
| | characteristics and by its effect on other hormones |
| | such as thyroid-stimulating hormone (TSH), |
| | melatonin, DHEA, IGF-1, and testosterone. |
| Impotence | enhances blood flow and improves hormonal |
| | functioning and utilization |
| Aging | enhances speed and efficiency of wound healing |
| Aging | enhances skin elasticity and thickness |
| Aging | facilitates hair regrowth and hair color restoration |
| | in some individuals |

hGH has been approved by the FDA for the treatment of growth hormone deficiency (GHD) in children and adults with a history of hypothalamic pituitary disease, short stature associated with chronic renal insufficiency before renal transplantations, short stature in patients with Turner syndrome or Prader-Willi syndrome, and infants born small for gestational age who have not caught up in height. Recently, hGH also has been approved for use in human immunodeficiency virus (HIV)-associated wasting in adults.

A stabilized macromolecular drug complex of the present invention provides improved treatment of such diseases and conditions by stabilizing the hGH, thereby increasing the amount of human growth hormone delivered to cells. A present stabilized macromolecular human growth hormone complex, when admixed with chitosan in a pharmaceutical formulation, can be administered by pulmonary delivery, and delivers more hGH to the cells than hGH alone because of enhanced bioavailability.

An important additional advantage of the present invention is to provide a method of administering a protein therapeutic, like insulin, human growth hormone, or other protein and polypeptide-based drugs, by pulmonary delivery. Such protein therapeutics, cannot be administered orally because the drug is altered in the stomach, and, therefore, is unavailable to the body in a form to combat or control a disease. Injections of insulin or hGH are useful therapeutically, but patient compliance often is compromised, especially in children.

With respect to diabetes, it is known that glucose can complex with proteins to produce toxic by-products. Such toxic by-products have been theorized as the cause of the complications associated with diabetes. It also has been observed that diabetics have elevated levels of GAGs in serum and urine, and a lower GAG content in their kidney cell membranes. It also is known that administration of GAGs to diabetic animals inhibited and/or reversed some vascular abnormalities associated with diabetes. Diabetics also have altered blood chemistries, including elevated levels of various enzymes in addition to glucose.

Therefore, the following has been hypothesized, but is not relied upon, as a cause for the complications associated with diabetes, and possibly other diseases. In particular, the interior of vascular walls are lined with endothelial cells. Branching from the endothelial cells are proteoglycan molecules. Glucose is able to bond with these surfaces of the endothelial cells. However, GAGs also are known to be present on the proteoglycan branches on the surface of endothelial cells. In addition, insulin and other therapeutic agents also are known to have the capability to complex with the GAG compounds. It is hypothesized, therefore, that insulin and other protein therapeutics complex with the GAGs present on the branches of the endothelial cells, and that the GAGs-drug complexes are removed from the cell by enzymatic activity, thereby leaving the surfaces endothelial cells devoid of GAGs compounds.

An increased drug dosage provides sufficient drug to account for the drug lost as a result of the insulin-GAGs interaction. But the sloughing of GAGs from endothelial cells exposes the vascular surface to numerous unwanted reactions, including repeated glycosylation. In addition, repeated glycosylation can be exacerbated by the naturally elevated levels of serum glucose in a diabetic. It has been found that the interaction between a protein therapeutic and the GAGs on the endothelial cells can be circumvented by complexing insulin, and other protein therapeutic, such that the protein therapeutic is unavailable to interact with the GAGs on the surface of endothelial cells.

It therefore was suggested to complex insulin with a GAG, and thereby protect vascular endothelial cells from the harmful effects of constant exposure to insulin, for example. Then, the insulin would not be available to complex with GAGs on the surface of endothelial cells. As a result, the endothelial cells would not be vulnerable to glycosylation as a result of a sloughing off of the GAGs-insulin complex. However, many high molecular weight GAGs are well known anticoagulants and their long term effects on a diabetic are unknown. As a result, a GAG, like heparin, could not be administered to an individual on a long term basis because, for example, the blood of the individuals would be thinned too greatly.

In accordance with the present invention, it has been shown that hGH, insulin, and other protein therapeutics, can be complexed with an excess stoichiometric molar amount of a polymer having a plurality of acid moieties, to provide a stabilized macromolecular drug complex that avoids the interaction between the protein therapeutic and a GAG on the surface of an endothelial cell. By utilizing an excess stoichiometric molar complexing of the polymer, the stability of the macromolecular drug complex is enhanced. It is hypothesized that the vascular endothelial cells, therefore, are spared from undesirable reactions, like glycosylation, and vascular complications associated with the disease or condition being treated can be eliminated or attenuated. Furthermore, a present stabilized macromolecular drug complex makes the protein therapeutic available to the individual, such that the disease or condition is controlled. Other protein therapeutics, in addition to hGH and insulin, also can be complexed with an excess of the stoichiometric complexing amount of the polymer, and made available to treat the disease or condition of concern.

The use of a suitable polymer also avoids the harmful side effects of GAGs (e.g., anticoagulation), and insures the quality, reproducibility, and uniformity of the stabilized macromolecular drug complex because the polymers have a reproducible chemical makeup, and the molecular weight can be controlled. Furthermore, by a proper selection of a polymer, the in vivo behavior of the protein therapeutic can be controlled to optimize the pharmacologic response of the protein therapeutic, and the route of administration can be regulated.

A protein therapeutic present in a present stabilized macromolecular drug complex can be any drug capable of complexing with a polymer having a plurality of acid moieties. Typically, the protein therapeutic has at least one positively charged site. The protein therapeutic is typically a naturally occurring drug, but synthetic protein therapeutics also can be used. The protein therapeutic is oligomeric or polymeric, like a polypeptide or protein. A protein therapeutic often contains an amino acid having a positively charged site. Such quaternized nitrogen atoms and positively charged sites are available to complex with the acid moieties of the polymer.

In accordance with the present invention, the term “protein therapeutic” includes (a) naturally occurring human proteins, including plasma proteins, (b) recombinant copies of naturally occurring proteins, (c) mutated and modified versions of a naturally occurring protein, and (d) monoclonal antibodies.

For example, if the drug is insulin, insulin contains fifty-one amino acids in two polypeptide chains. The insulin molecule contains the amino acids lysine, arginine, and histidine. Each of these amino acids has a positively charged site, thereby permitting insulin to complex with the polymer through the acid moieties of the polymer. Similarly, human growth hormone contains 191 amino acids in one polypeptide chain. Human growth hormone also contains the amino acids lysine, arginine, and histidine, which, like insulin, contain positively charged sites thereby permitting the growth hormone to complex with the polymer through the acid moieties of the polymer. It should be understood that derivatives of human growth hormone containing 190 or 192 amino acids, and hydrolysis products of human growth hormone that behave identically or similarly to human growth hormone, are encompassed by the term “human growth hormone” as used herein. Suitable forms of hGH include, but are not limited to, pituitary hGH (pit-hGH), methionyl hGH (met-hGH), and recombinant hGH (rhGH).

Other protein therapeutics also can be complexed with a stoichiometric excess molar amount of a polymer having a plurality of acid moieties to form a stabilized macromolecular drug complex of the present invention. These protein therapeutics include, but are not limited to, polymyxin, bacitracin, tuberactionomycin, ethryomycin, penicillamine, glucosamine, an interferon (e.g., interferon α, β, or γ), albumin, elcatonin, granulocyte colony stimulating factor (GCSF), transforming growth factor-beta 2 (TGF-β2), erythropoietin, immune globulin, glucocerebrosidase, factor VIII, factor IX, fibrin, follicle stimulating hormone, tissue necrosis factor, factor VIIa, hepatitis B immune globulin, growth releasing factor, secretin, LHRH, acidic fibroblast growth factor (a-FGF), keratinocyte growth factor (KGF), growth hormone releasing hormone, bradykin antagonists, enkephalins, nifedipin, THF, insulin-like growth factors, atrial natriuretic peptide, vasopressin, ACTH analogs, and glucagon. Monoclonal antibodies useful as the protein therapeutic include, but are not limited to, muromonab-CD3, abciximab, edrecolomab, rituximab, daclizumab, trastuzumab, palivizumab, basiliximab, and infliximab.

The polymer used to prepare the macromolecular drug complex has a plurality of acid moieties. Any physiologically acceptable polymer can be used as long as the polymer contains sufficient acid moieties to complex with the drug. Typically, the polymer has sufficient acid moieties if the polymer can be solubilized in water by neutralizing the polymer with a base. The polymer typically is a naturally occurring polymer, but synthetic counterparts and derivatives of a naturally occurring polymer also can be used, as can synthetic polymers. In general, the polymer has an M_{w} of about 2,000 to about 50,000, and preferably about 5,000 to about 45,000. To achieve the full advantage of the present invention, the polymer has an M_{w} of about 10,000 to about 20,000.

With respect to naturally occurring polymers, the above-discussed disadvantages resulting from using a GAG limits the naturally occurring polymers to those that do not adversely effect an individual over the long term, i.e., a strong anticoagulant should not be used as the polymer. However, GAGs that act as anticoagulants have a relatively high molecular weight of about 12,000 or greater. Therefore, analogs of GAGs that do not act as strong anticoagulants can be used as the polymer. Such polymers have a structure that is similar to a GAG compound.

Dermatan sulfate (DS) also is a GAG. DS having an M_{w} ranging from 12 to 45 kDa, is a polydisperse, linear copolymer consisting of N-acetyl-D-galactopyranose, L-iodopyranosyluronic acid, and D-glucopyranosyluronic acid. DS routinely is prepared commercially from porcine and bovine intestinal mucosa or porcine skin. DS has important anticoagulant and antithrombotic activities, and the anticoagulant effect of DS is about 70-fold less potent that heparin on a per weight basis. Thus, the hemorrhagic properties of DS are greatly reduced when compared to those of heparin. DS has a relatively short half-life and low bioavailability, compared to heparin delivered by subcutaneous or intramuscular routes.

Therefore, useful naturally occurring polymers have an M_{w} of about 5,000 to about 45,000, and preferably about 10,000 to about 20,000, and do not act as coagulants at the level they are present in the macromolecular drug complex. The dose of macromolecular drug complex, e.g., about 2 mg/day, is less than the 20 mg/day dose required to observe anticoagulation effects and, therefore, mild anticoagulants can be used as the polymer. Furthermore, the low M_{w}, naturally occurring polymers have a greater bioavailability. For example, heparin having an M_{w} of about 6,000 is 85% bioavailable, but as the M_{w} increases, bioavailability decreases exponentially. Suitable naturally occurring polymers, therefore, include, but are not limited to, heparin, dermatan sulfate, chondroitin sulfate, keratan sulfate, heparin sulfate, hyaluronic acid, the various forms of carrageenan, and mixtures thereof, having a molecular weight of about 4 to about 8,000 kDa.

Synthetic polymers also are useful in the preparation of a macromolecular dry complex of the present invention. Such synthetic polymers include, but are not limited to, polystyrene sulfonate, polyacrylic acid, and polyvinylphosphonic acid. Additional synthetic polymer having a plurality of acid moieties are disclosed in U.S. Pat. No. 6,417,234, incorporated herein by reference.

The following experiments illustrate the improved stabilization achieved by utilizing a molar excess of the polymer when complexing the protein therapeutic. These experiments are directed to stabilized macromolecular drug compositions containing hGH as the protein therapeutic, but other protein therapeutics are envisioned as behaving similarly. Additional procedures, experimental data, and discussion are presented in Appendix A.

In particular, hGH is a protein hormone essential for normal growth and developments in humans. hGH is susceptible to a variety of degradation processes, which make it an unstable protein. hGH is a complex protein that forms insoluble adducts with heparin (see U.S. Pat. No. 6,417,237). Because the hGH/UH adducts are insoluble, the adducts are theorized to be more stable than the hGH alone. However, it now has been demonstrated that hGH is more stable as the adduct in the presence of a stoichiometric molar excess of the soluble heparin (i.e., unfractionated heparin, UH). It is theorized, but not relied upon, that stabilization is achieved by stabilization of the native, folded structure of hGH and the heparin acts as a protecting agent to reduce protein-protein interactions that result in denaturation and aggregation. This result is unexpected in the case of a large and complicated protein structure.

Stability Studies

a) Interfacial Denaturation Aggregation Method

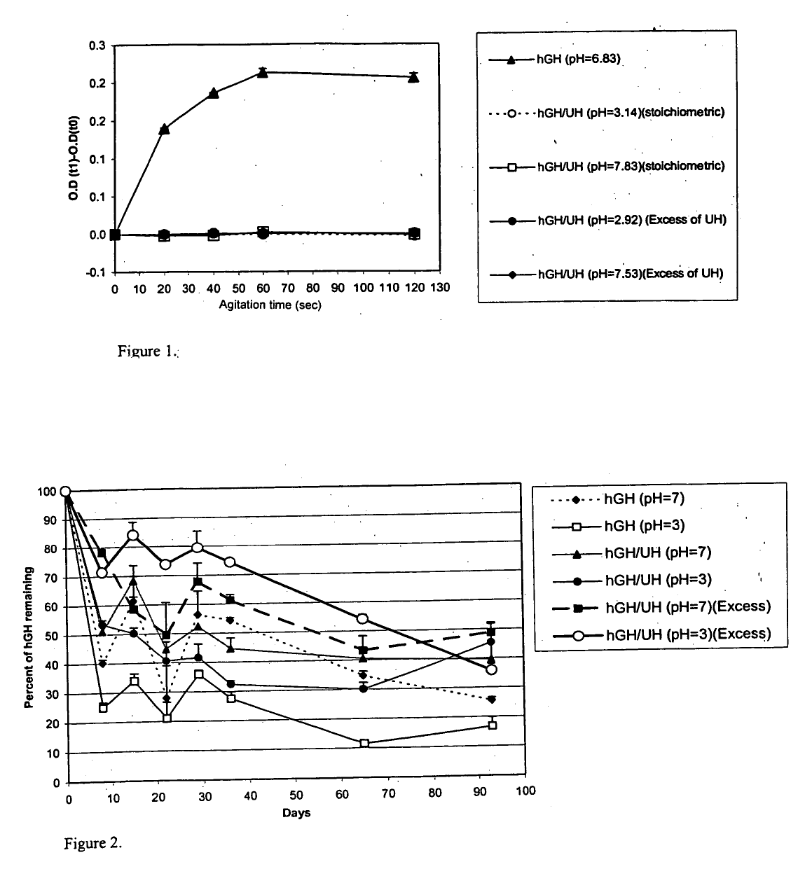

Shear-induced aggregation is the most common degradation process for hGH. Therefore, a high air-water interface was introduced into the sample vortex agitation, as a comparative denaturing technique, to induce aggregation. This technique was applied to hGH and hGH/UH complexes (0.5 mg/ml hGH) at different pH values, then optical densities (at 450 nm) were determined by UV spectrophotometry. An increase in optical density (OD) is an indication of protein aggregation. The test results showed that vortex agitation over 120 seconds resulted in no changes in the optical density at 450 nm of hGH/UH adducts (for both stoichiometric and excess amounts of heparin) compared to a substantial increase for hGH alone (FIG. 1).

b) Real-Time Stability Studies

Real-time stability studies of hGH/UH adducts also were performed at different pH values (i.e., pH=3 and 7) and temperatures (4° C. and 37° C.) for 93 days. hGH was quantified by ELISA (enzyme linked immunosorbent assay).

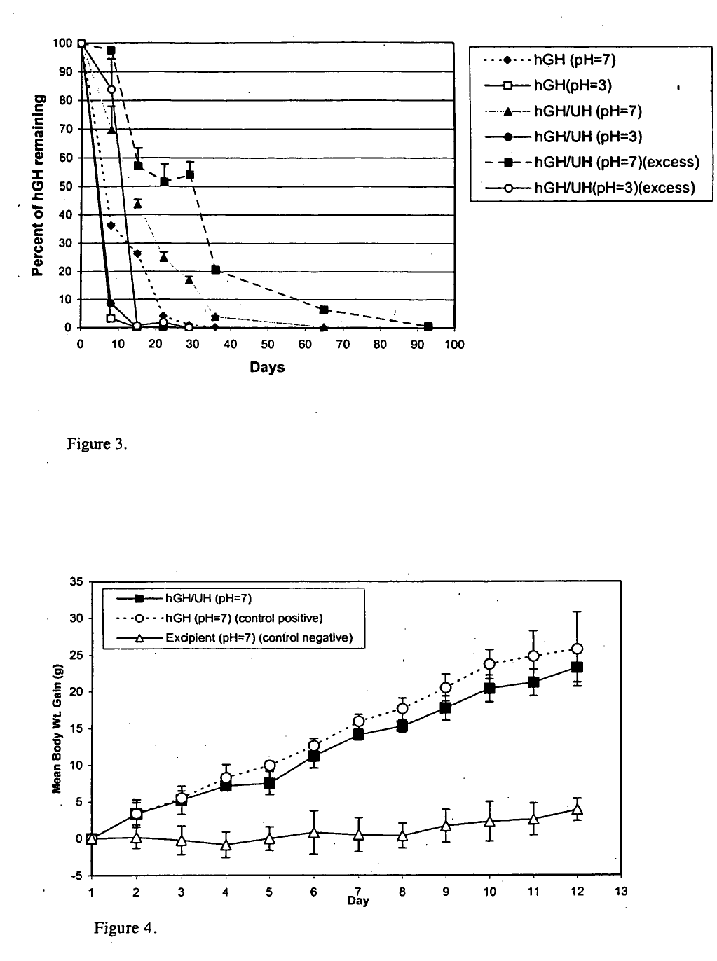

The test results showed that hGH/UH adducts with an excess of heparin (pH=3 and 7) have the highest percent of hGH remaining in solution or, alternatively stated, were the most stable formulations (FIGS. 2 and 3).

The real-time stability studies provided a better validation of stable formulation (i.e., three months).

In Vivo Studies

The hypophysectomized (hypox) female rat body weight gain (BWG) bioassay is presently the most widely used bioassay, and has been termed the defining bioassay, for hGH to assess biopotency and biological activity of hGH (Bangham et al., 1985; USP Pharmacopeial Forum, 1990). The bioassay end point, i.e., BWG, and its long duration provide an excellent model of the clinical or veterinary circumstances for which hGH is used.

hGH/UH complexes, with and without excess heparin, were prepared at pH=3 and lyophilized. The amount of heparin in excess was four times greater than the stoichiometric amount. On the day of administration, the adducts were reconstituted in phosphate buffered saline (pH=7.4) to simulate physiological body conditions. hGH/UH with excess of heparin at pH=7 and 37° C. (body temperature) was the most stable hGH formulation in vitro.

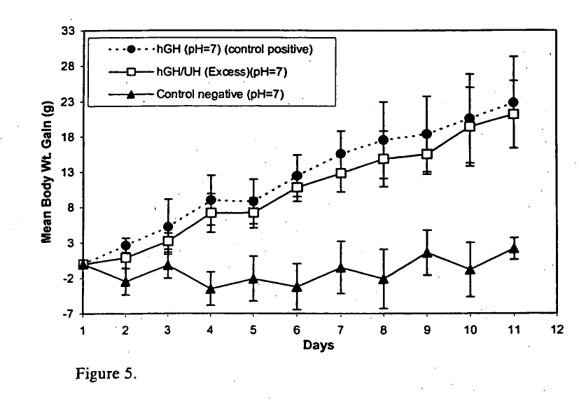

Weight gain studies in female hypophysectomized rats indicated equivalent biological activity of hGH/UH complexes to hGH via subcutaneous and intratracheal administration over an 11- or 10-day period, respectively (FIGS. 4 and 5). For FIG. 4, the cumulative BWG was for female rats administered daily about equivalent doses of hGH (0.32 mg/kg), subcutaneously for 11 days. For FIG. 5, the cumulative BWG was for female rats administered alternate daily about equivalent doses of hGH (2.5 mg/kg), intratracheally for 10 days. Complexation of hGH with UH did not affect the growth-promoting activity of hGH.

In summary, an increase in the stability of hGH in the presence of an excess stoichiometric molar complexing amount of heparin, without significant changes in biological activity of hGH, has been observed.

Further tests showed that complex formation is optimized by adding the polymer to the protein therapeutic, and by using minimal agitation or stirring to mix the reactants. Good complex formation however was observed when the protein therapeutic was added to the polymer with minimal or no agitation or stirring.

Different weight ratios of hGH to heparin were used to prepare the macromolecular drug complexes. A stoichiometric ratio of protein therapeutic to polymer is defined as the presence of neither an excess protein therapeutic or an excess polymer in a filtrate of a protein therapeutic-polymer complex. A stoichiometric molar amount of hGH to heparin is 1.8:1, alternatively 70:30 on a weight basis. To achieve improved stability the amount of heparin is increased over this stoichiometric amount. Therefore, about two molecules of hGH interact with one heparin molecule to provide a stoichiometric hGH-heparin adduct.

A present macromolecular complex has a mole ratio of hGH to heparin is at least about 1.8:1.5. The mole ratio of hGH to heparin can be as high as about 1.8:8. A preferred mole ratio of hGH to heparin is about 1.8:2.5 to about 1.8:6. To achieve the full advantage of the present invention, the hGH to heparin mole ratio is about 1.8:3 to about 1.8:5. Accordingly, an excess molar amount of heparin over the stoichiometric molar amount required to complex with hGH is used, and a macromolecular drug complex of improved stability is achieved.

Similarly, for other polymers and protein therapeutics, the molar amount of the polymer in the complex is in excess of that required to stoichiometrically complex with the protein therapeutic. The molar stoichiometric amount of polymer and protein therapeutic is easily determined by persons skilled in the art, and is related to the identity of the polymer and protein therapeutic. For example, the molar stoichiometric amount can be determined by preparing hGH/heparin suspensions using twelve different molar ratios. Each of the twelve samples is passed through a 20 nm anodisc filter membrane. The filtrate is analyzed for starting materials, i.e., hGH by ELISA and heparin by the standard azure A dye binding method.

A suspension of a solid, stabilized macromolecular complex is formed by complexing a protein therapeutic with a stoichiometric molar excess of the free acid form of the polymer. In particular, a solution of the protein therapeutic is combined with an aqueous solution of the acid form of the polymer, and a precipitate forms. This precipitate, i.e., the stabilized macromolecular dry complex, is insoluble in aqueous media at an acidic pH.

After formation of the stabilized macromolecular drug complex, the complex is isolated (if necessary), then incorporated into a pharmaceutical formulation. The stabilized macromolecular drug complex is relatively hydrophobic, and, therefore, has a tendency to concentrate in the oil phase of the formulation, e.g., the dispersed phase of an oil-in-water emulsion or the continuous phase in a water-in-oil emulsion. The presence of the stabilized macromolecular drug complex in the oil phase has advantages, e.g., the protein therapeutic is less susceptible to hydrolysis and oxidation. Pharmaceutical formulations containing a macromolecular drug complex can be prepared as set forth in U.S. Pat. No. 6,417,237, incorporated herein by reference, and by using methods and ingredients known to persons skilled in the art.

For example, a stabilized macromolecular drug complex can be formulated in suitable excipients and vehicles for oral, parenteral, or pulmonary administration. Such excipients are well known in the art. The stabilized macromolecular drug complex typically is present in such a pharmaceutical formulation in an amount of about 0.1% to about 75% by weight.

Pharmaceutical formulations containing a stabilized macromolecular drug complex of the present invention are suitable for administration to humans or other mammals. Typically, the pharmaceutical formulations are sterile, and contain no toxic, carcinogenic, or mutagenic compound which would cause an adverse reaction when administered.

The stabilized macromolecular drug complex can be administered by any suitable route, for example by oral, buccal, inhalation, sublingual, rectal, vaginal, intracisternal through lumbar puncture, transurethral, nasal, or parenteral (including intravenous, intramuscular, subcutaneous, and intracoronary) administration. Parenteral administration can be accomplished using a needle and syringe. Implant pellets also can be used to administer a nanoparticle drug composition parenterally. The stabilized macromolecular drug complex also can be administered as a component of an ophthalmic drug-delivery system. As disclosed more fully hereafter, a present stabilized macromolecular complex also is useful for pulmonary delivery when the pharmaceutical formulation contains chitosan.

The pharmaceutical formulations include those wherein the stabilized macromolecule drug complex is administered in an effective amount to achieve its intended purpose. More specifically, a “therapeutically effective amount” means an amount effective to treat a disease. Determination of a therapeutically effective amount is well within the capability of those skilled in the art, especially in light of the detailed disclosure provided herein.

The exact formulation, route of administration, and dosage is determined by an individual physician in view of the patient's condition. Dosage amount and interval can be adjusted individually to provide levels of the stabilized macromolecular drug complex that are sufficient to maintain therapeutic or prophylactic effects.

The amount of pharmaceutical formulation administered is dependent on the subject being treated, on the subject's weight, the severity of the affliction, the manner of administration, and the judgment of the prescribing physician.

Specifically, for administration to a human in the curative or prophylactic treatment of a disease, oral dosages of the stabilized macromolecular drug complex is about 10 to about 500 mg daily for an average adult patient (70 kg). Thus, for a typical adult patient, individual doses contain about 0.1 to about 500 mg stabilized macromolecular drug complex, in a suitable pharmaceutically acceptable vehicle or carrier, for administration in single or multiple doses, once or several times per day. Dosages for intravenous, buccal, or sublingual administration typically are about 0.1 to about 10 mg/kg per single dose as required. In practice, the physician determines the actual dosing regimen that is most suitable for an individual patient and disease, and the dosage varies with the age, weight, and response of the particular patient. The above dosages are exemplary of the average case, but there can be individual instances in which higher or lower dosages are merited, and such are within the scope of this invention.

A stabilized macromolecular drug complex of the present invention can be administered alone, or in admixture with a pharmaceutical carrier selected with regard to the intended route of administration and standard pharmaceutical practice. Pharmaceutical formulations for use in accordance with the present invention, including ophthalmic preparations, thus can be formulated in a conventional manner using one or more physiologically acceptable carriers comprising excipients and auxiliaries that facilitate processing of a stabilized macromolecular drug complex into preparations that can be used pharmaceutically.

These pharmaceutical formulations can be manufactured in a conventional manner, e.g., by conventional mixing, dissolving, granulating, dragee-making, emulsifying, or lyophilizing processes. Proper formulation is dependent upon the route of administration chosen. When a therapeutically effective amount of the stabilized macromolecular drug complex is administered orally, the formulation typically is in the form of a tablet, capsule, powder, solution, or elixir. When administered in tablet form, the formulation additionally can contain a solid carrier, such as a gelatin or an adjuvant. The tablet, capsule, and powder contain about 5% to about 95%, preferably about 25% to about 90%, of a stabilized macromolecular drug complex of the present invention. When administered in liquid form, a liquid carrier, such as water, petroleum, or oils of animal or plant origin, can be added. The liquid form of the pharmaceutical formulation can further contain physiological saline solution, dextrose or other saccharide solutions, or glycols. When administered in liquid form, the pharmaceutical formulation contains about 0.5% to about 90%, by weight, of a stabilized macromolecular drug complex, and preferably about 1% to about 50%, by weight, of a stabilized macromolecular drug complex.

When a therapeutically effective amount of a stabilized macromolecular drug complex is administered by intravenous, cutaneous, or subcutaneous injection, the composition is in the form of a pyrogen-free, parenterally acceptable aqueous preparation. The preparation of such parenterally acceptable solutions, having due regard to pH, isotonicity, stability, and the like, is within the skill in the art. A preferred preparation for intravenous, cutaneous, or subcutaneous injection typically contains an isotonic vehicle in addition to a stabilized macromolecular drug complex of the present invention.

A stabilized macromolecular drug complex can be readily combined with pharmaceutically acceptable carriers well-known in the art. Such carriers enable the stabilized macromolecular drug complex to be formulated as tablets, pills, dragees, capsules, liquids, gels, syrups, slurries, suspensions and the like, for oral ingestion by a patient to be treated. Pharmaceutical preparations for oral use can be obtained by adding the nanoparticle drug composition with a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A stabilized macromolecular drug complex can be formulated for parenteral administration by injection, e.g., by bolus injection or continuous infusion. Preparations for injection can be presented in unit dosage form, e.g., in ampules or in multidose containers, with an added preservative. The preparations can take such forms as suspensions, solutions, or emulsions in oily or aqueous vehicles, and can contain formulatory agents such as suspending, stabilizing, and/or dispersing agents.

Pharmaceutical formulations for parenteral administration include aqueous dispersions of the stabilized macromolecular drug complex. Additionally, suspensions of the stabilized macromolecular drug complex can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the dispersibility of the compounds and allow for the preparation of highly concentrated preparations. Alternatively, a present pharmaceutical formulation can be in powder form for constitution with a suitable vehicle, e.g., sterile pyrogen-free water, before use.

A stabilized macromolecular drug complex also can be formulated in rectal compositions, such as suppositories or retention enemas, e.g., containing conventional suppository bases. In addition to the preparations described previously, the stabilized macromolecular drug complex also can be formulated as a depot preparation. Such long-acting preparations can be administered by implantation (for example, subcutaneously or intramuscularly) or by intramuscular injection. Thus, for example, the stabilized macromolecular drug complex can be formulated with suitable polymeric or hydrophobic materials (for example, as an emulsion in an acceptable oil) or ion exchange resins.

In particular, the stabilized macromolecular drug complex can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A formulation also can be injected parenterally, for example, intravenously, intramuscularly, subcutaneously, or intracoronarily. For parenteral administration, the formulation is best used in the form of a sterile aqueous solution which can contain other substances, for example, salts, or monosaccharides, such as mannitol or glucose, to make the solution isotonic with blood.

For veterinary use, the stabilized macromolecular drug complex is administered as a suitably acceptable formulation in accordance with normal veterinary practice. The veterinarian can readily determine the dosing regimen and route of administration that is most appropriate for a particular animal.

In particular, the pulmonary absorption of a stabilized macromolecular complex of the present invention is enhanced by incorporating chitosan in a pharmaceutical formulation containing the stabilized macromolecular drug complex. As previously stated, hGH is a protein hormone essential for normal growth and development in humans. hGH is administered by subcutaneous injections mainly to growth hormone deficient children, daily or six times per week. Pulmonary delivery of hGH as an alternative route for hGH administration to overcome the pain and inconvenience of injections has been investigated. The overall goal of this research is to find a useful form of hGH for pulmonary delivery. The present stabilized hGH/UH (unfractionated heparin) adducts in a pharmaceutical formulation further containing chitosan provides a useful composition for the pulmonary delivery of hGH.

Chitosan is a known mucoadhesive and has been reported to enhance absorption of a number of materials across cellular membranes. It now has been discovered that the biological activity of human growth hormone, when complexed and stabilized with excess heparin, is enhanced in the presence of small quantities of chitosan, but diminished by larger quantities of chitosan. Chitosan did not improve the pulmonary adsorption of uncomplexed hGH. This discovery suggests that chitosan plays a significant role in the pulmonary absorption of the hGH-heparin complex.

Preparation of Chitosan Microparticles and hGH/UH Loading

Chitosan microparticles were prepared by the method of Tian et al. (1999), which is an adaptation of the method described by Berthold et al. (1996). Briefly, chitosan was dissolved in 2% (v/v) acetic acid containing 1% (v/v) TWEEN® 80. The resulting solution was transferred into a sonication bath, then stirred at 414 rpm with a blade stirrer. Sodium sulfate solution (20%, w/v) was added dropwise during sonication with stirring (for 30 minutes) to a final sodium sulfate concentration of about 0.66% (w/v). Then, 5.0 ml of 0.25% glutaraldehyde solution was added, and sonication and stirring was continued for another hour. Crosslinking was quenched by addition of 100 ml of 12% (w/v) sodium metabisulfite solution. The formed chitosan microparticles were recovered by superspeed centrifuge (5000 rpm, 15 min), then washed twice with double distilled water. The average particle size of the chitosan microparticles was about 1 μm to about 1.5 μm. Typically, the particles have an average size of about 1 to about 5 μm to allow for pulmonary delivery. The surfaces of the chitosan microparticles were positively charged, e.g., about 10 to about 20 millivolts in water.

A suspension of stabilized hGH/UH adduct (pH=3) with an excess stoichiometric molar amount of heparin was admixed with an aqueous suspension of the chitosan microparticles (pH=3.7) at different weight ratios. The resulting mixtures were maintained at room temperature with constant shaking by an orbit shaker at 150 rpm for 1 hour followed by lyophilization. Then, on the day of administration, the formulations were reconstituted in phosphate buffered saline (pH=7.4) to simulate physiological body conditions.

In Vivo Studies

The hypophysectomized (hypox) female rat body weight gain (BWG) bioassay was used to assess biopotency and biological activity of hGH (Bangham et al., 1985; USP Pharmacopeial Forum, 1990). The bioassay end point, BWG, and its long duration provides an excellent model of the clinical or veterinary circumstance under which hGH is used.

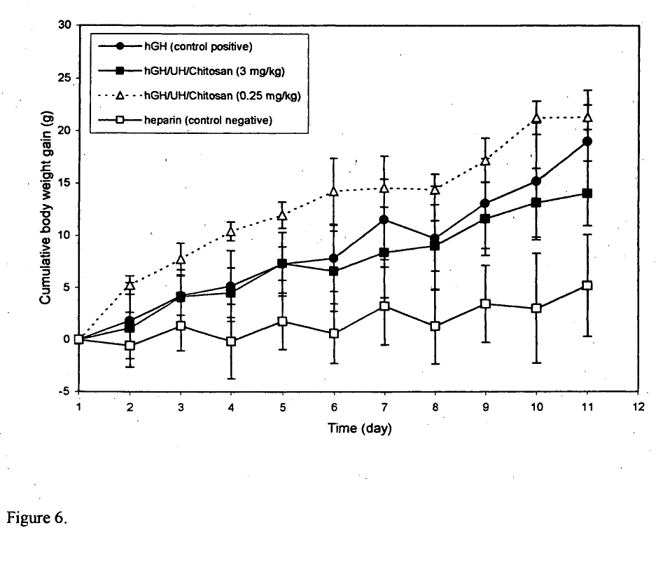

In vivo studies demonstrated substantially higher cumulative body weight gain for pharmaceutical formulations containing a present hGH/UH complex and chitosan (i.e., 2.5 mg hGH/kg—4.2 mg heparin/kg, plus 0.25 mg chitosan/kg body weight) than hGH alone, when administered intratracheally on alternate days over ten days (FIG. 6). However, this result was not observed for all quantities of chitosan. FIG. 6 shows cumulative BWG for hypophysectomized rats (5 to 6 per group) given alternate daily intratracheal administration of heparin alone, hGH alone (2.5 mg/kg), hGH complexes (2.5 mg hGH/kg and 4.2 mg heparin/kg) with 0.25 mg chitosan/kg or 3 mg chitosan/kg, for 10 days.

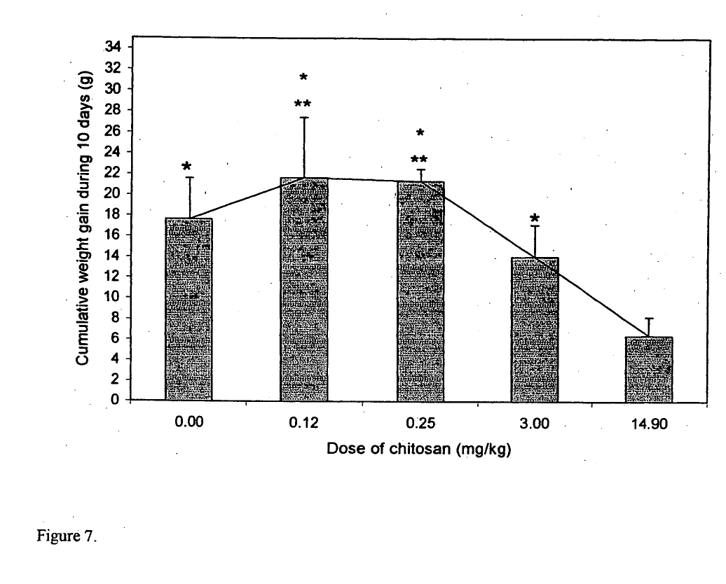

Test results showed an approximately bell-shaped distribution in weight gain by changing the quantities of chitosan microparticles, and a decrease in weight gain at higher quantities of chitosan (FIG. 7). FIG. 7 shows the effect of amount of chitosan on normalized cumulative BWG of hypophysectomized rats over 10 days. The rats were administered alternate daily intratracheal instillation of hGH complexes (2.5 mg hGH/kg and 4.2 mg hGH/mg heparin) plus four doses of chitosan particles (0, 0.12, 0.25, 3, and 14.9 mg chitosan/kg).

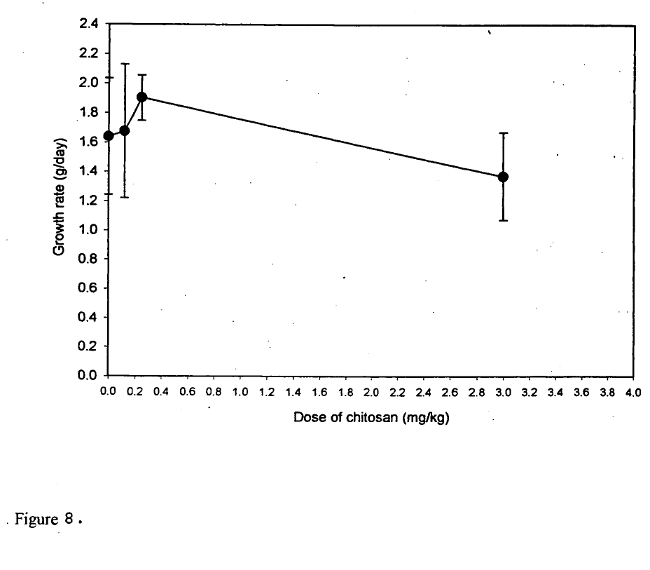

Growth rate was another property used to compare hGH formulations. Growth rate was calculated from the slope of weight gain curves versus days (FIG. 8). FIG. 8 shows the effect of chitosan amount of normalized growth rate of hypophysectomized rats treated with alternate daily instillations of intratracheal hGH complexes (2.5 mg hGH/kg and 4.2 mg heparin/kg) plus three doses of chitosan microparticles (0, 0.12, 0.25, and 3 mg chitosan/kg) over 10 days. hGH/UH/chitosan (2.5 mg hGH/kg—4.2 mg heparin/kg) plus 0.25 mg chitosan/kg body weight, produced highest growth rates for the different quantities of chitosan tested.

In summary, chitosan has a positive effect on the absorption of hGH complexed and stabilized with excess heparin through the mucus membranes of the lungs. Moreover, the effect of chitosan follows approximately a bell-shaped distribution resulting in an increase in the absorption at low amounts of chitosan and a decrease in absorption in higher amounts of chitosan. With regard to weight gain and growth rate results, hGH/UH/chitosan (2.5 mg hGH/kg-4.2 mg heparin/kg), plus 0.25 mg chitosan/kg body weight, was found to be the optimum formulation for the pulmonary administration of hGH.

Improved pulmonary absorption of hGH in a present stabilized complex is observed when chitosan is administered in an amount of about 0.01 to about 2 mg/kg, and preferably about 0.03 to about 1 mg/kg. To achieve the full advantage of the present invention, chitosan is administered in an amount of about 0.05 to about 0.75 mg/kg.

Although the present disclosure is particularly directed to the preparation of stabilized macromolecular hGH-heparin complexes, persons skilled in the art can apply this disclosure to a variety of protein therapeutics capable of complexing with a polymer having a plurality of acid moieties, e.g., heparin. The complexes are prepared by simply admixing an excess stoichiometric molar complexing amount of the polymer, preferably in the free acid form, with the protein therapeutic in an aqueous medium. The specific physicochemical properties of the resulting macromolecular complex can be adjusted by a judicious selection of the polymer and the M_{w} of the polymer, by the number and type of acid moieties on the polymer, by the mole ratio of protein therapeutic to polymer in the macromolecular complex, and by the number and type of polymer crosslinks.

For example, tests were performed to demonstrate that polymers in addition to heparin can be complexed with a protein therapeutic to provide a stable macromolecular drug complex of the present invention. In these tests, hGH was complexed with other polymers both to illustrate the scope of the invention, and to find a complexing polymer that avoids the potential adverse effects associated with a long-term administration of heparin. In particular, despite the effectiveness of heparin-induced stabilization of hGH, heparin is an inherently heterogeneous compound and possesses well-known anticoagulant activity. A polymer that demonstrates the stabilizing capabilities of heparin, and having no or reduced adverse side effects, therefore, would be beneficial.

In this test, two polymers, one from the glycosaminoglycan (GAG) family (i.e., dermatan sulfate, DS) and the second outside the GAG family (i.e., polystyrene sulfonate, PSS), were evaluated for binding to hGH.

Heparin is the highest negatively charged polymer among the GAGs. DS has the second highest binding strength when interacting with proteins. However, the anticoagulant effect of DS is about 70-fold less than heparin on a weight basis. Thus, the hemorrhagic side effects of DS are greatly reduced compared to heparin. Experiments were performed to investigate an interaction between hGH and the polymer and the stoichiometric ratio of hGH/polymer adducts.

Dermatan sulfate (chondroitin sulfate B) sodium salt from bovine mucosa was purchased from Sigma, St. Louis, Mo. Sodium polystyrene sulfonate (M_{w}=16.8 kDa) was obtained from Polymer Standards Service GmbH, Mainz, Germany.

hGH/DS complexes and hGH/PSS complexes were prepared by the same method described above for the hGH/heparin (UH) complexes (acidic method). The stoichiometric ratio of the hGH/DS and hGH/PSS complexes was determined as discussed above for the hGH/UH complexes.

Dermatan sulfate and polystyrene sulfonate were found to interact with hGH with stoichiometric ratios of 70:30 and 80:20 (w:w) hGH/polymer, respectively. The molar stoichiometric ratios of hGH/polymer complexes were found to be 2.7:1 and 3.1:1 for DS and PSS, respectively. Due to heterogeneity of dermatan sulfate, i.e., a reported molecular weight range of 12 to 45 dKa, an average molecular weight of 25 kDa was assumed in order to calculate the molar ratio of hGH/DS complexes at the stoichiometric ratio.

This test illustrates that the interaction of a protein therapeutic, like hGH, with a polymer is not limited to heparin, but also occurs with other polyanionic polymers. More similarities were observed between UH and DS complexes, which is expected because these polymers both belong to the same family of glycosaminoglycans. PSS is outside of GAG family, but still possesses an ability to interact quantitatively with hGH.

Therefore, many modifications and variations of the invention as hereinbefore set forth can be made without departing from the spirit and scope thereof, and only such limitations should be imposed as are indicated by the appended claims.

### References

N. B. Bam et al., Journal of Pharmaceutical Sciences, 87, 1554-1559 (1998);
D. R. Bangham et al., Molecular and Cellular Endocrinology, 42, 269-282 (1985);
M. Katakam et al., Pharmaceutical Development and Technology, 2, 143-149 (1997);
Martindale, The Extra Pharmacopoeia. Royal Pharmaceutical Society, London (1996).
R. Pearlman et al., In Y. J. Wang et al. (eds.), Stability and characterization of protein and peptide drugs: Case histories, Plenum Press, New York, 1-58 (1993);
J. A. Schroeder-Tefft et al., Journal of Controlled Release, 49, 291-298 (1997);
H. H. Sorensen et al., U.S. Pat. No. 6,022,858 (2000).
USP Pharmacopeial Forum, The United States Pharmacopeial Convention, Rockville, Md., 1246-1264 (1990);
K. Arai et al., Bull Tokai Reg Fish Res Lab Japan, 56, 89-94 (1968);
P. Artursson et al., Pharm. Res., 11, 1358-1361 (1994);
A. Berthold et al., J. Control Release, 39, 17-25 (1996);
S. D. Frasier, Endocr. Rev., 4, 155-170 (1983);
P. He et al., Int. J. Pharm., 166, 75-68 (1998);
L. Illum, U.S. Pat. No. 5,690,954 (1997);
L. Illum, U.S. Pat. No. 5,863,554 (1999);
L. Illum et al., Pharm Res, 11, 1186-9 (1994);
J. S. Patton, Adv Drug Deliv Rev, 19, 3-36 (1996);
J. S. Patton et al., Adv Drug Deliv Rev, 8, 179-196 (1992);
J. S. Patton et al., Biotechnology Therapeutics, 1, 213-228 (1989-1990);
A. K. Singla et al., J. Pharm Pharmacol, 53, 1047-1067 (2001);
S. S. Steiner et al., U.S. Pat. No. 6,428,771 (2002);
X. Tian et al., J Pharm Pharmacol, 51, 151-157 (1999);
C. Witschi et al., Pharm Res, 16, 382-90 (1999);
H. Yamamoto et al., Proceed Int'l Symp Control Rel Bioact Mater, 1018-1019 (2000).

1. A macromolecular drug complex comprising:
(a) a protein therapeutic; and
(b) a polymer having a plurality of acid moieties and a weight average molecular weight of about 2,000 to about 50,000,
said complex having a mole ratio of the polymer to the protein therapeutic in excess of a stoichiometric molar amount required to complex the polymer and the protein therapeutic.

2. The complex of claim 1 wherein the protein therapeutic is selected from the group consisting of a naturally occurring human protein, a recombinant copy of a naturally occurring protein, a mutated or modified version of a naturally occurring protein, a monoclonal antibody, and mixtures thereof.

3. The complex of claim 1 wherein the protein therapeutic is a polypeptide or a protein.

4. The complex of claim 1 wherein the protein therapeutic is selected from the group consisting of insulin, human growth hormone, polymyxin, bacitracin, tuberactionomycin, ethryomycin, penicillamine, glucosamine, glucagon, interferon α, interferon β, interferon γ, albumin, elcatonin, granulocyte colony stimulating factor, transforming growth factor-beta 2, erythropoietin, immune globulin, glucocerebrosidase, factor VIII, factor IX, fibrin, follicle stimulating hormone, tissue necrosis factor, factor VIIa, hepatitis B immune globulin, growth releasing factor, secretin, LHRH, acidic fibroblast growth factor, keratinocyte growth factor, growth hormone releasing hormone, bradykin antagonists, enkephalins, nifedipin, THF, insulin-like growth factors, atrial natriuretic peptide, vasopressin, an ACTH analog, and mixtures thereof.

5. The complex of claim 1 wherein the protein therapeutic is a monoclonal antibody selected from the group consisting of muromonab-CD3, abciximab, edrecolomab, rituximab, daclizumab, trastuzumab, palivizumab, basiliximab, infliximab, and mixtures thereof.

6. The complex of claim 1 wherein the protein therapeutic is insulin.

7. The complex of claim 1 wherein the protein therapeutic is a human growth hormone.

8. The complex of claim 7 wherein the human growth hormone is selected from the group consisting of pit-hGH, met-hGH, rhGH, and mixtures thereof.

9. The complex of claim 1 wherein the polymer has a weight average molecular weight of about 5,000 to about 45,000.

10. The complex of claim 1 wherein the polymer is in a free acid form.

11. The complex of claim 1 wherein the polymer comprises a naturally occurring polymer having a weight average molecular weight of about 10,000 to about 20,000.

12. The complex of claim 11 wherein the naturally occurring polymer is selected from the group consisting of heparin, dermatan sulfate, chondroitin sulfate, keratan sulfate, heparin sulfate, hyaluronic acid, carrageenan, and mixtures thereof.

13. The complex of claim 1 wherein the polymer comprises a synthetic polymer.

14. The complex of claim 13 wherein the polymer is selected from the group consisting of polystyrene sulfonate, polyacrylic acid, polyvinyl-phosphonic acid, and mixtures thereof.

15. The complex of claim 1 wherein the polymer comprises heparin.

16. The complex of claim 15 wherein the protein therapeutic comprises a human growth hormone.

17. The complex of claim 16 wherein the molar ratio of the human growth hormone to the heparin is about 1.8:1.5 to about 1.8:8.

18. The complex of claim 16 wherein the molar ratio of the human growth hormone to the heparin is about 1.8:2.5 to about 1.8:6.

19. The complex of claim 1 wherein the protein therapeutic is insulin or a human growth hormone, and the polymer comprises heparin.

20. The complex of claim 19 wherein the heparin has a weight average molecular weight of about 10,000 to about 20,000.

21. A pharmaceutical composition comprising:
(a) a macromolecular drug complex of claim 1; and
(b) chitosan.

22. The composition of claim 21 wherein the chitosan is in a form of microparticles.

23. The composition of claim 22 wherein the chitosan microparticles have an average particle size of about 1 μm to about 5 μm.

24. A method of treating a disease or a condition comprising administering to a mammal in need thereof a therapeutically effective amount of a pharmaceutical composition containing a human growth hormone complex to an individual, said human growth hormone complex comprising: (a) a human growth hormone, and (b) a polymer having a plurality of acid moieties and a weight average molecular weight of about 2,000 to about 50,000, said complex having a mole ratio of the polymer to the human growth hormone in excess of a stoichiometric molar amount required to complex the polymer and the human growth hormone.

25. The method of claim 24 wherein the disease or condition is selected from the group consisting of dwarfism, hypopituitarism, hypercholesterolemia, depression, muscle wasting, osteoporosis, insomnia, menopause, impotence, hypothalamic pituitary disease, short stature associated with chronic renal insufficiency before renal transplantations, short stature associated with Turner syndrome or Prader-Willi syndrome, HIV-associated wasting, and a condition associated with aging.

26. The method of claim 25 wherein the composition is administered intravenously.

27. The method of claim 24 wherein the mammal is a human.

28. A method of treating a disease or a condition comprising administering to a mammal in need thereof a therapeutically effective amount of a pharmaceutical formulation containing a human growth hormone complex and chitosan to an individual, said human growth hormone complex comprising: (a) a human growth hormone, and (b) a polymer having a plurality of acid moieties and a weight average molecular weight of about 2,000 to about 50,000, said complex having a mole ratio of the polymer to the human growth hormone is excess of a stoichiometric molar amount required to complex the polymer and the human growth hormone.

29. The method of claim 28 wherein the disease or condition is selected from the group consisting of dwarfism, hypoptititarism, hypercholesterolemia, depression, muscle wasting, osteoporosis, insomnia, menopause, impotence, hypothalamic pituitary disease, short stature associated with chronic renal insufficiency before renal transplantations, short stature associated with Turner syndrome or Prader-Willi syndrome, HIV-associated wasting, and a condition associated with aging.

30. The method of claim 28 wherein the chitosan is in a form of microparticles.

31. The method of claim 28 wherein the chitosan is administered in an amount of about 0.01 to about 2 mg/kg of body weight of the mammal.

32. The method of claim 28 wherein the mammal is a human.

33. The method of claim 28 wherein the pharmaceutical formulation is administered via pulmonary delivery.

34. A method of treating diabetes in an individual comprising administering a therapeutically-effective amount of a composition containing a macromolecular drug complex of claim 1, wherein the protein therapeutic of the complex comprises insulin, to a diabetic.

35. The method of claim 34 wherein the composition is administered intravenously.

36. The method of claim 34 wherein the composition is administered orally.

37. The method of claim 34 wherein the composition further comprises chitosan.

38. The method of claim 37 wherein the composition is administered via pulmonary delivery.
